(19) United States
(12) Patent Application Publication
(10) Pub. No.: US 2009/0112122 A1
(43) Pub. Date: Apr. 30, 2009

(54) BLEEDING APPARATUS

(76) Inventors: Min-Chieh Chuang, Tainan City (TW); Herbert Ho, Taipei City (TW)

Correspondence Address: NORTH AMERICA INTELLECTUAL PROPERTY CORPORATION, P.O. BOX 506, MERRIFIELD, VA, 22116, US

(21) Appl. No.: 12/124,156

(22) Filed: May 21, 2008

(30) Foreign Application Priority Data
Oct. 26, 2007 (TW) 096140383

(51) Int. Cl. A61B 5/15

(52) U.S. Cl. 600/583

(57) ABSTRACT

A bleeding apparatus includes a stimulating component for stimulating an examinee on a first region, and a bleeding component for bleeding the examinee on a second region. The second region is adjacent to a boundary of the first region.

### BACKGROUND OF THE INVENTION

1. Field of the Invention

The present invention relates to a bleeding apparatus and, more particularly, to a bleeding apparatus capable of reducing pain of an examinee when bleeding the examinee.

2. Description of the Prior Art

Recently patients can use portable examining instruments to examine themselves due to the progress of medical technology. For example, a diabetic can examine blood sugar by himself at home with a bleeding needle for collecting blood. Because the bleeding needle pricks to subcutaneous nociceptors of his skin, the examinee has obvious sensation of pain. It causes an obstacle of self-examination especially for chronic patients who need to use bleeding needles often. The patients repel bleeding due to sensation of pain causing health deterioration indirectly.

FIG. 1 illustrates a schematic view of a conventional bleeding apparatus 10. The bleeding apparatus 10 includes a rod 12. A contacting surface 14 is disposed on an end of the rod 12. An opening 16 is disposed on the contacting surface 14 so that a needle 18 installed inside the rod 12 is capable of protruding therethrough. The contacting surface 14 of the bleeding apparatus 10 contacts with skin of a user when bleeding the user. The needle 18 is capable of protruding through the opening 16 when turning on a switch (not shown in FIG. 1) on the rod 12. However, the user has obvious sensation of pain when the needle 18 pricks skin. There is a need to design a bleeding apparatus capable of reducing an examinee's pain when bleeding the examinee.

### SUMMARY OF THE INVENTION

It is therefore a primary objective of the present invention to provide a bleeding apparatus capable of reducing pain of an examinee when bleeding the examinee.

According to the present invention, a bleeding apparatus includes a stimulating component for stimulating an examinee on a first region, and a bleeding component for bleeding the examinee on a second region. The second region is adjacent to a boundary of the first region.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

The present invention will become more fully understood from the detailed description given herein below and the accompanying drawings which are given by way of illustration only, and thus are not limitative of the present invention, and wherein:

FIG. 1 illustrates a schematic view of a conventional bleeding apparatus.

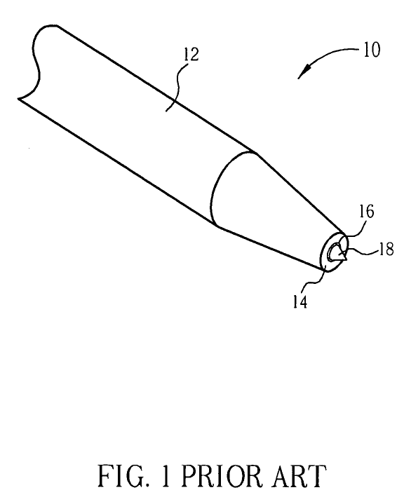

FIG. 2 illustrates a diagram of lateral inhibition in human perception physiology.

FIG. 3 illustrates a lateral view of a bleeding apparatus according to a first embodiment of the present invention.

FIG. 4 illustrates a diagram of the bleeding apparatus bleeding an examinee according to the first embodiment of the present invention.

FIG. 5 illustrates a lateral view of a bleeding apparatus according to a second embodiment of the present invention.

FIG. 6 illustrates a diagram of the bleeding apparatus bleeding the examinee according to the second embodiment of the present invention.

### DETAILED DESCRIPTION

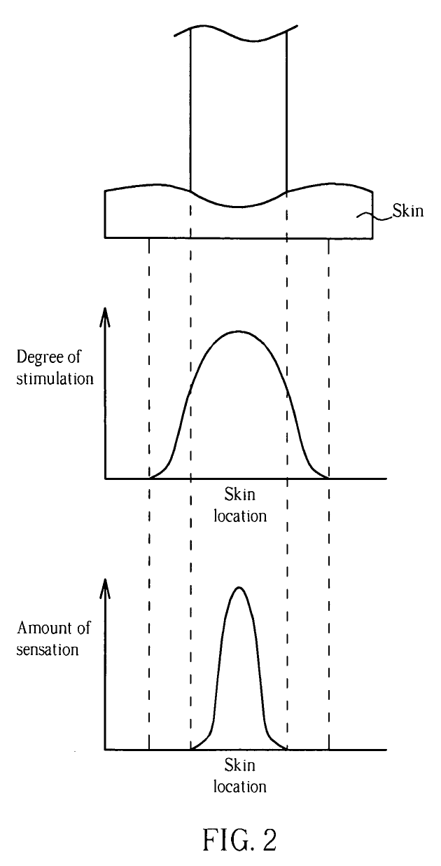

FIG. 2 illustrates a diagram of lateral inhibition in human perception physiology. When a blunt column presses human skin as illustrated above, the degree of stimulation of human skin is presented as the curve illustrated in middle. The degree of stimulation corresponding to the center of the column is the largest, and the degree of stimulation is decreasing outwardly. The range of stimulation is larger than the section of the column slightly. However the sensation range of human central nervous system is less than that of real stimulation and centralizes. It is so-called lateral inhibition.

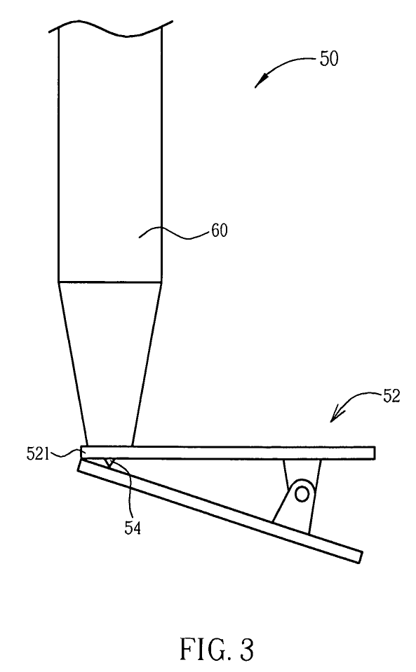

FIG. 3 illustrates a lateral view of a bleeding apparatus 50 according to a first embodiment of the present invention. As shown in FIG. 3, the bleeding apparatus 50 includes a stimulating component 52, and a bleeding component 54 for pricking an examinee's skin. The bleeding component 54 can be a bleeding needle. The stimulating component 52 can be a fixture for stimulating the examinee's skin by clamping. The bleeding component 54 is disposed on a clamping side 521 of the stimulating component 52.

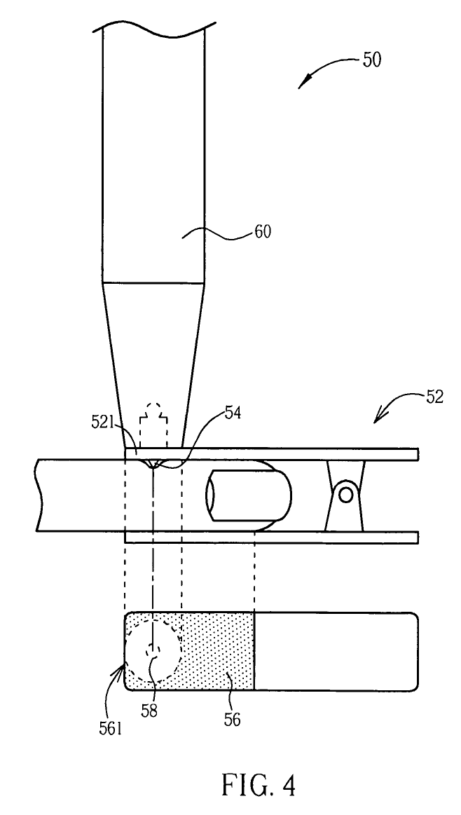

FIG. 4 illustrates a diagram of the bleeding apparatus 50 bleeding the examinee according to the first embodiment of the present invention. The stimulating component 52 can clamp the examinee's finger. A first region 56 is defined as the lateral clamping region of the finger. The first region 56 includes a boundary 561, and the bleeding component 54 is disposed within a second region 58 adjacent to the boundary 561 and corresponding to a designated range inwardly from the boundary 561 of the first region 56. The designated range can correspond to a quarter or the other ratio of the square measure of the first region 56. When the stimulating component 52 clamps the examinee's finger laterally, the degree of stimulation presents as the curve illustrated in middle of FIG. 2. The degree of stimulation corresponding to the center of the first region 56 is largest, and the degree of stimulation decreases gradually and outwardly. Due to lateral inhibition, the sensation range of the examinee is less than the range of real stimulation and sharpens in a central part of the first region 56 clamped by the stimulating component 52 laterally, and peripheral stimulation can not sensed by the examinee easily. For this reason, the bleeding component 54 is disposed at the second region 58, which is near the boundary 561. When the stimulating component 52 clamps the examinee's finger, the bleeding component 54 bleeds the examinee's finger simultaneously. At this time, the examinee is not easy to sense pain due to the bleeding component 54 pricking the examinee's skin at the second region 58 so that the pain can be reduced. In addition, the bleeding component 54 can be disposed inside a body 60 in an extendable manner and is capable of protruding out of the body 60 when turning on a switch (not shown in the drawings) on the body 60 for bleeding the examinee. The bleeding component 54 also can be disposed on an end of the body 60 directly for bleeding the examinee directly.

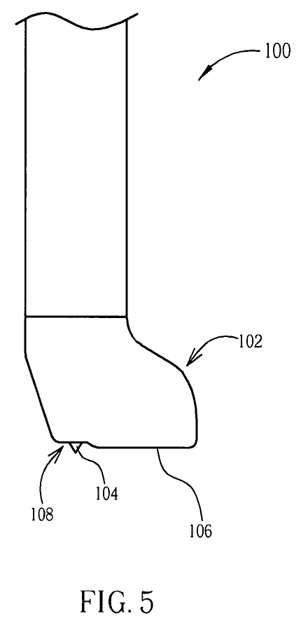

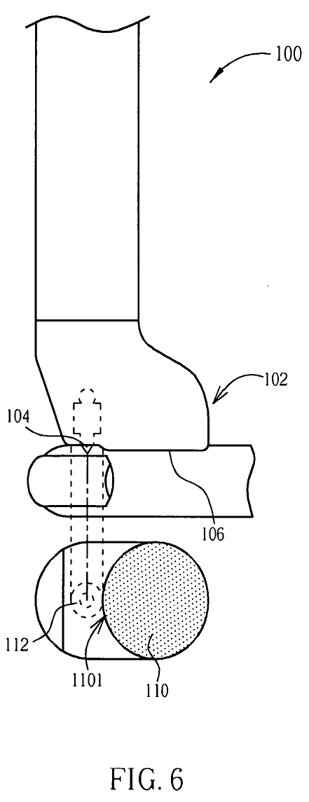

FIG. 5 illustrates a lateral view of a bleeding apparatus 100 according to a second embodiment of the present invention. The bleeding apparatus 100 includes a stimulating component 102, and a bleeding component 104 for bleeding blood out of the examinee's skin. The stimulating component 102 can be a rod-type structure, and a stimulating plane 106 is formed on an end of the stimulating component 102. The stimulating plane 106 can be a circular cross section, a rectangular cross section, or other shapes. A recess 108 is formed adjacent to a side of the stimulating plane 106, and the bleeding component 104 is disposed inside the recess 108. FIG. 6 illustrates a diagram of the bleeding apparatus 100 bleeding the examinee. The structure of the stimulating component 102 is designed for holding easily. The user can hold the stimulating component 102 to simulate the examinee's finger. A first region 110 is defined corresponding to the stimulating plane 106 as a stimulating region of the finger. The range of the first region 110 is substantially equal to that of the stimulating plane 106. The first region 110 includes a boundary 1101, and the bleeding component 104 is disposed within a second region 112 outside the first region 110 and adjacent to the boundary 1101. That is, the recess 108 is formed on the second region 112. The second region 112 corresponds to a designated range corresponding to a quarter or other ratio of square measure of the first region 110. When the stimulating plane 106 of the stimulating component 102 presses the examinee's finger, the degree of stimulation presents as the curve illustrated in middle of FIG. 2. The degree of stimulation corresponding to the center of the stimulating plane 106 is largest, and the degree of stimulation decreases gradually and outwardly. Due to lateral inhibition, the sensation range of the examinee is less than the range of real stimulation and sharpens in a central part of the first region 110 stimulated by the stimulating component 102, and the peripheral stimulation is hard to be sensed by the examinee easily. For this reason, the bleeding component 104 is disposed at the recess 108 on the rim of the first region 110, the second region 112. When the stimulating component 102 stimulates the examinee's finger, the bleeding component 104 bleeds the examinee's finger simultaneously. At this time, the examinee's human central nervous system can not sense pain due to the bleeding component 104 pricking the examinee's skin easily at the second region 112 so that the pain can be reduced. In addition, the bleeding component 104 can be disposed inside the rod-type structure in an extendable manner and is capable of protruding out of the rod-type structure when turning on a switch (not shown in the drawings) on the rod-type structure for bleeding the examinee. The recess 108 can be disposed selectively, and the bleeding component 104 can be disposed on the stimulating plane 106 in an extendable manner. The bleeding component 104 also can be disposed on an end of the rod-type structure directly. The user presses the stimulating component 102 directly, and the stimulating plane 106 of the stimulating component 102 stimulates the examinee's finger first. Then the bleeding component 104 inside the recess 108 pricks the examinee's skin for bleeding the examinee with increasing pressure.

In conclusion, the bleeding apparatus of the present invention stimulates the examinee's skin with the stimulating component first and then bleeds the examinee on the rim of the stimulating region with the bleeding component. Due to lateral inhibition, the examinee can not sense pain of bleeding obviously so as to reduce the examinee's pain when bleeding the examinee. The stimulation of the stimulating component can be presented in a contact manner such as pressure sensation, touch sensation or temperature sensation. The stimulation of the stimulating component also can be presented in a non-contact manner such as laser stimulation. The structural and disposition design of the stimulating component and the bleeding component is not limited to above-mentioned embodiments, and any mechanical design capable of reaching lateral inhibition effect is within the scope of the present invention.

Compared with the prior art, the bleeding apparatus of the present invention stimulates the examinee's skin with the stimulating component first and then bleeds the examinee with the bleeding component near the boundary of the stimulating region. Due to lateral inhibition, the examinee can not sense pain of bleeding obviously so as to reduce the examinee's pain when bleeding the examinee.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A bleeding apparatus comprising:
a stimulating component having a first region for stimulating an examinee; and
a bleeding component for bleeding the examinee at a second region adjacent to a boundary of the first region.

2. The bleeding apparatus of claim 1, wherein the stimulating component is a fixture having a clamping side and the bleeding component is disposed on the clamping side.

3. The bleeding apparatus of claim 1, wherein the bleeding component is a needle.

4. The bleeding apparatus of claim 1, wherein the stimulating component comprises a stimulating plane, a range of the stimulating plane is equal to that of the first region, a recess is formed on a side of the stimulating plane, and the bleeding component is disposed inside the recess.

5. The bleeding apparatus of claim 4, wherein the bleeding component is disposed inside the recess in an extendable manner.

6. The bleeding apparatus of claim 4, wherein the stimulating component is a rod-type structure.

7. The bleeding apparatus of claim 4, wherein the stimulating plane has a circular cross section.

8. The bleeding apparatus of claim 4, wherein the stimulating plane has a rectangular cross section.

9. The bleeding apparatus of claim 1, wherein the second region corresponds to a range of a quarter of the first region inwardly from the boundary of the first region.

10. The bleeding apparatus of claim 1, wherein the second region corresponds to a range of a quarter of the first region outwardly from the boundary of the first region.

11. The bleeding apparatus of claim 1, wherein the bleeding component is disposed in a body in an extendable manner.

12. The bleeding apparatus of claim 11, wherein the bleeding component is capable of protruding out of the body when turning on a switch on the body.

13. The bleeding apparatus of claim 1, further comprising a stimulating plane formed on an end of the stimulating component, and the bleeding component is disposed on the stimulating plane in an extendable manner.

14. The bleeding apparatus of claim 1, wherein the stimulating component is disposed on a terminal of a rod-type structure.

15. The bleeding apparatus of claim 1, wherein the bleeding component is disposed on an end of a body.
